(19) United States
(12) Patent Application Publication
(10) Pub. No.: US 2025/0041372 A1
(43) Pub. Date: Feb. 6, 2025

(54) Pharmaceutical composition for antibacterial wound healing comprising chia seeds and biodegradable materials

(71) Applicant: UIF (University Industry Foundation), Yonsei University, Seoul (KR)

(72) Inventors: Yong SHIN, Seoul (KR); Zhen QIAO, Seoul (KR)

(21) Appl. No.: 18/776,013

(22) Filed: Jul. 17, 2024

(30) Foreign Application Priority Data
Aug. 1, 2023 (KR) 10-2023-0100501

(51) Int. Cl. A61K 36/537; A61K 47/10; A61K 47/26; A61K 47/32; A61P 17/02; A61P 31/04

(52) U.S. Cl. CPC A61K 36/537; A61K 47/10; A61K 47/26; A61K 47/32; A61P 17/02; A61P 31/04

(57) ABSTRACT

Proposed is a pharmaceutical composition for antibacterial wound healing, the pharmaceutical composition including chia seeds and biodegradable substances. The pharmaceutical composition may be customized depending on the wound and may also be prepared in a transparent form. The pharmaceutical composition exhibits unique self-adhesive properties, so the pharmaceutical composition may be directly attached to the skin and withstand joint movement through excellent deformation adaptability and has high tensile strength. The pharmaceutical composition also has an antibacterial effect and an excellent wound-healing effect.

### CROSS REFERENCE TO RELATED APPLICATION

The present application claims priority to Korean Patent Application No. 10-2023-0100501, filed Aug. 1, 2023, the entire contents of which is incorporated herein for all purposes by this reference.

### BACKGROUND OF THE DISCLOSURE

### 1. Field of the Disclosure

The present disclosure relates to a pharmaceutical composition for antibacterial wound healing and, more specifically, to a pharmaceutical composition for antibacterial wound healing, the pharmaceutical composition including chia seeds and biodegradable substances.

### 2. Description of the Related Art

Chia seeds are edible seeds of Salvia hispanica, which is an annual herbaceous plant in the Lamiaceae family. Chia seeds are rich in omega-3 and omega-6 fatty acids, vitamins, antioxidants, and minerals. These nutritional properties of chia seeds have been widely verified and used in the food industry. Meanwhile, the shells of fully hydrated chia seeds release anionic heteropolysaccharides and have high water retention capacity and high viscosity and thickening effects. Thus, chia seed mucilage may be used as a thickening agent, gelling agent, and chelating agent in foods and pharmaceuticals. However, currently, research on chia seeds is limited to foods, and chia seed mucilage as a promising emerging biomaterial has not yet been developed.

Polyvinyl alcohol (PVA) is a non-toxic water-soluble polymer obtained by hydrolyzing polyvinyl acetate. Hydrogels made from PVA have excellent mechanical properties, controllability, and degradability, but have poor biocompatibility compared to natural polymers. The properties of the prepared hydrogels may be improved by proportionally mixing PVA with other natural polymers.

In some studies, preparation of electrospinning and film products has been proposed by mixing PVA with plant seed mucilage, including chia seed mucilage. However, most of the studies focus on mechanical characterization and subsequent applications in the food and packaging sectors.

Meanwhile, the skin is the largest surface of the human body that comes into contact with the external environment, and the skin takes up the largest area in the human body. The skin is the first line of defense that protects the body from physical, chemical, mechanical damage, and biological invasion from the external environment. Wounds are generally formed when external damaging factors act on the skin surface. As the prevalence of chronic diseases increases, chronic wounds such as skin ulcers, bedsores, and diabetic foot, which are side effects caused by the use of drugs, are also showing a high incidence. In wounds, there are usually varying degrees of contamination due to rupture and bleeding of the skin and mucosa in the injured area, and the wound area is prone to infection.

Hydrogels currently available on the market for wound care mainly have a structure of a thin layer of polyurethane coated with an adhesive on one side and a moisture-resistant film on the other. Additional adhesives may cause skin reactions or allergic reactions in people with sensitive skin. When the adhesives are applied to the wound for a long time, the area may be sticky when the adhesives are removed, and the long-term use of the adhesives may cause further external damage to the wound area. At the same time, commercial hydrogel films may protect the wound from external contaminants. However, these physical methods may not be suitable for infected wounds because bacteria may be trapped, resulting in healing delay. Additionally, since the methods are currently more expensive than other wound dressing methods, it is disadvantageous that the methods increase the burden of medical expenses on patients.

Accordingly, there is a need to develop a wound dressing agent that has self-adhesive properties, is hypoallergenic, has an antibacterial effect, and has a wound-healing effect that may be produced at low cost by using easily available raw materials.

### SUMMARY OF THE DISCLOSURE

Therefore, the present disclosure is to provide a pharmaceutical composition for antibacterial wound healing, the pharmaceutical composition including chia seeds and biodegradable substances.

1. A pharmaceutical composition for antibacterial wound healing, the pharmaceutical composition including chia seeds and biodegradable substances.

2. The pharmaceutical composition for antibacterial wound healing of 1 described above, in which the chia seeds are included in one or more forms selected from the group consisting of hydrated chia seed shells, swollen chia seeds, and chia seed mucilage.

3. The pharmaceutical composition for antibacterial wound healing of 1 described above, in which the biodegradable substances include polyvinyl alcohol (PVA), sorbitol, and glycerol.

4. The pharmaceutical composition for antibacterial wound healing of 2 described above, in which the chia seed mucilage is contained in an amount of 0.5 to 2 w/v %.

5. The pharmaceutical composition for antibacterial wound healing of 3 described above, in which the PVA is contained in an amount of 0.5 to 2 w/v %.

6. The pharmaceutical composition for antibacterial wound healing of 3 described above, in which the sorbitol is contained in an amount of 1.5 w/v %.

7. The pharmaceutical composition for antibacterial wound healing of 3 described above, in which the glycerol is contained in an amount of 1.5 v/v %.

8. A wound dressing agent including the pharmaceutical composition of 1 described above.

9. The wound dressing agent of 8 described above, in which the wound dressing agent is formulated into one or more types selected from the group consisting of film, cream, gel, and spray.

10. A cosmetic composition including chia seeds and biodegradable substances.

11. A quasi-drug composition including chia seeds and biodegradable substances.

The present disclosure relates to a pharmaceutical composition for antibacterial wound healing, the pharmaceutical composition including chia seeds and biodegradable substances. The pharmaceutical composition can be customized depending on the wound and can also be prepared in a transparent form. The pharmaceutical composition exhibits unique self-adhesive properties, so the pharmaceutical composition can be directly attached to the skin and withstand joint movement through excellent deformation adaptability and has high tensile strength. The pharmaceutical composition also has an antibacterial effect and an excellent wound-healing effect.

However, the effect of the disclosure is not limited to the effects mentioned above and can be clearly understood by those skilled in the art from other effects not mentioned.

### BRIEF DESCRIPTION OF THE DRAWINGS

FIG. 1 shows a schematic diagram of the preparation process of a pharmaceutical composition for antibacterial wound healing, the pharmaceutical composition including chia seeds and biodegradable substances;

FIG. 2 shows a photograph showing the results of preparing a film-type wound dressing agent depending on the content of chia seed mucilage and PVA;

FIG. 3 shows a photograph showing the swelling degree of the film-type wound dressing agent in PBS depending on the content of chia seed mucilage and PVA;

FIG. 4 shows a graph showing the swelling height ratio of the film-type wound dressing agent depending on the content of chia seed mucilage and PVA;

FIG. 5 shows photographs showing the transparency, adhesive properties, and deformation adaptability of the film-type wound dressing agent, respectively;

FIG. 6 shows photographs showing an experiment to confirm the tensile strength and peel strength of the film-type wound dressing agent;

FIG. 7 shows graphs showing the swelling ratio and decomposition rate of the film-type wound dressing agent over time, respectively;

FIG. 8 shows photographs and graphs showing the antibacterial effects of the pharmaceutical composition for wound healing;

FIG. 9 shows a graph showing the biocompatibility of the pharmaceutical composition for wound healing;

FIG. 10 shows photographs and a graph showing scratch analysis results of the pharmaceutical composition for wound healing;

FIG. 11 shows a photograph showing the wound healing results of the film-type wound dressing agent;

FIG. 12 shows a graph showing the analysis results of the wound healing area of the film-type wound dressing agent;

FIG. 13 shows photographs showing the thickness evaluation of the epithelial layer in the wound area through hematoxylin-eosin staining analysis;

FIG. 14 shows photographs showing further analysis of the distribution of inflammatory cells in the wound area and regeneration of dermal tissue by using high-resolution histology microscopy;

FIG. 15 shows graphs showing quantitative analysis of inflammatory cells and collagen in the wound area, respectively;

FIG. 16 shows graphs showing relative quantitative analysis of mRNA expression levels in the wound area on days 3, 10, and 14; and

FIG. 17 shows photographs and graphs showing immunohistochemical staining for actin (α-SMA), macrophage marker F4/80, and transforming growth factor beta (TGFβ) marker at the wound area and the density of each protein, respectively.

### DESCRIPTION OF THE PREFERRED EMBODIMENTS

Hereinafter, the pharmaceutical composition for antibacterial wound healing, the pharmaceutical composition including chia seeds and biodegradable substances according to the present disclosure, and a method of preparing the same composition will be described in detail.

The present disclosure relates to a pharmaceutical composition for antibacterial wound healing, the pharmaceutical composition including chia seeds and biodegradable substances.

In the present disclosure, chia seeds may be edible seeds of Salvia hispanica or Salvia columbariae. The chia seeds may contain mucilage released from the shells of hydrated chia seeds.

The mucilage may be an anionic heteropolysaccharide. The mucilage may include α-D-xylan, β-D-glucopyranose, and 4-O-methyl-D-glucuronic acid. The catechol quinone structure of the polysaccharide-based compounds may exhibit adhesive properties by interacting with the sulfhydryl group functional group and cysteine group on the skin surface. In addition, the polysaccharide-based compounds may suppress or destroy biofilm formation by inhibiting the attachment of bacterial cells and forming a polymer membrane layer on the cell surface, causing internal metabolic disorders in bacteria.

Additionally, the mucilage may be obtained by centrifuging hydrated chia seeds.

In addition, the pharmaceutical composition for wound healing according to the present disclosure may contain the mucilage at an amount of 0.5 w/v %, 1 w/v %, and 2 w/v %, but the concentration is not limited thereto.

In the present disclosure, the biodegradable substances may be PVA but are not limited thereto. PVA is a non-toxic water-soluble polymer obtained by hydrolyzing polyvinylacetate and may have various degrees of polymerization.

In addition, the pharmaceutical composition for wound healing of the present disclosure may contain the PVA at an amount of 0.5 w/v %, 1 w/v %, and 2 w/v %, but the concentration is not limited thereto.

In the present disclosure, the biodegradable substances may contain a plasticizer. The plasticizer increases the flexibility, ductility, plasticity, and processability of a composition. The plasticizer may be sorbitol and glycerol but is not limited thereto. The carboxyl group in the chia seed mucilage polysaccharide chain and the PVA hydroxyl group are subjected to self-assembly and cross-linking under weak acid conditions, forming covalent hydrogen bonds. Due to that, a solution state may be made viscous.

In addition, the pharmaceutical composition for wound healing of the present disclosure may contain the sorbitol at an amount of 1 w/v % and glycerol at an amount of 1 v/v %, but the concentrations are not limited thereto.

The present disclosure may be a wound dressing agent containing a pharmaceutical composition for wound healing. In addition, the wound dressing agent may be formulated by selecting any one type from the group consisting of film, cream, gel, and spray but is not limited thereto.

The present disclosure may be a cosmetic composition or a quasi-drug composition including chia seeds and biodegradable substances but is not limited thereto.

Hereinafter, the present disclosure will be explained in detail by examples. The following examples illustrate the present disclosure, and the content of the present disclosure is not limited to the following examples.

### 1. Preparation of Chia Seed Mucilage

Chia seed mucilage is obtained through a hydration process. Chia seeds were mixed with distilled water in a glass beaker at a weight ratio of 1:30. Next, the mixture was stirred on a continuous magnetic stirrer (TMM-5, TOPSCIEN Instrument Co., Ltd.) for 3 hours at room temperature to ensure complete swelling and finally to induce the release of the mucilage of the chia seeds. The swollen chia seeds and the released mucilage were centrifuged for 60 minutes at a 5394×g centrifugal force using a centrifuge (1236R, LABOGENE Instrument Co., Ltd.). The mucilage in the upper layer obtained by centrifugation was collected and filtered through 200-mesh cheesecloth. After removing the mucilage in the upper layer, the swollen chia seeds in the lower layer were also squeezed out and filtered through a 200-mesh filter bag, thereby the maximum amount of chia seed mucilage on the surface thereof was secured. The extracted chia seed mucilage was freeze-dried and stored at room temperature.

### 2. Preparation of Pharmaceutical Compositions for Wound Healing and Film-Type Wound Dressing Agents

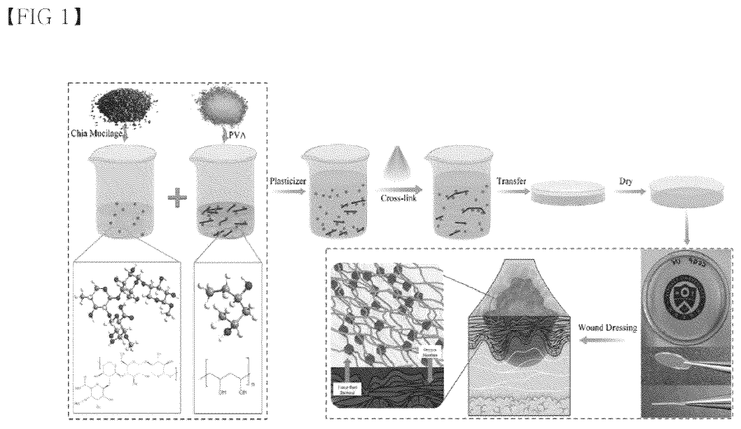

The freeze-dried chia seed mucilage was added to distilled water and completely dissolved at a temperature of 37° C. using a magnetic stirrer. Sorbitol (pre-dissolved in distilled water) and glycerin were then added to the rehydrated chia seed mucilage. The PVA solution was added when the chia seed mucilage, sorbitol, and glycerin were completely dissolved and mixed. Pharmaceutical compositions for wound healing were prepared by mixing the mixture and the PVA solution at 500 rpm for 10 minutes. Then, 25 ml of each composition was poured into cell culture dishes with a diameter of 9 cm. Next, the dishes were moved to a super-clean bench and subjected to UV irradiation for 60 minutes. After the cross-linking sterilization, the cell culture dishes containing each composition were subjected to drying in an oven at a temperature of 56° C. for 12 hours. The preparation of film-type wound dressing agents (Preparation Examples 1 to 9) was confirmed (FIG. 1). The content of chia seed mucilage and PVA for each formulation is shown in Table 1 below.

TABLE 1

| | Preparation Example | | | | | | | | |
|---|---|---|---|---|---|---|---|---|---|
| | 1 | 2 | 3 | 4 | 5 | 6 | 7 | 8 | 9 |
| Chia seed | 0.5 | 0.5 | 0.5 | 1 | 1 | 1 | 2 | 2 | 2 |
| mucilage (w/v %) | | | | | | | | | |
| PVA (w/v %) | 0.5 | 1 | 2 | 0.5 | 1 | 2 | 0.5 | 1 | 2 |

| | |
|---|---|
| Glycerin (v/v %) | 1.5 |
| Sorbitol (w/v %) | 1.5 |
| Volume (ml) | 25 |

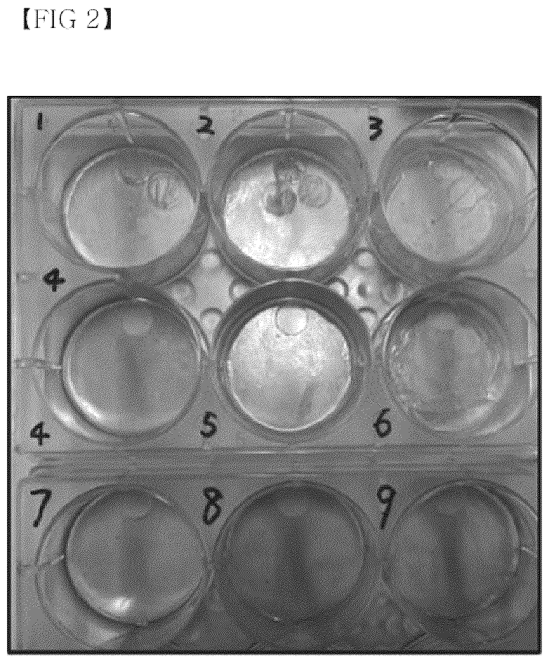

In the synthesis of Preparation Example 1 using 0.5% chia seed mucilage and 0.5% PVA, a film could not be formed after drying since there were not enough polymer chains to support synthesis. However, Preparation Example 1 was in a wax state and could not be effectively separated from the mold (FIG. 2).

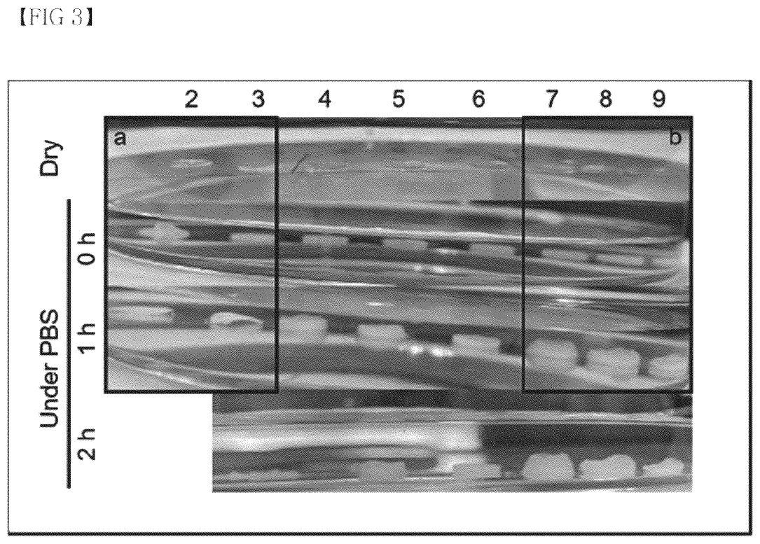

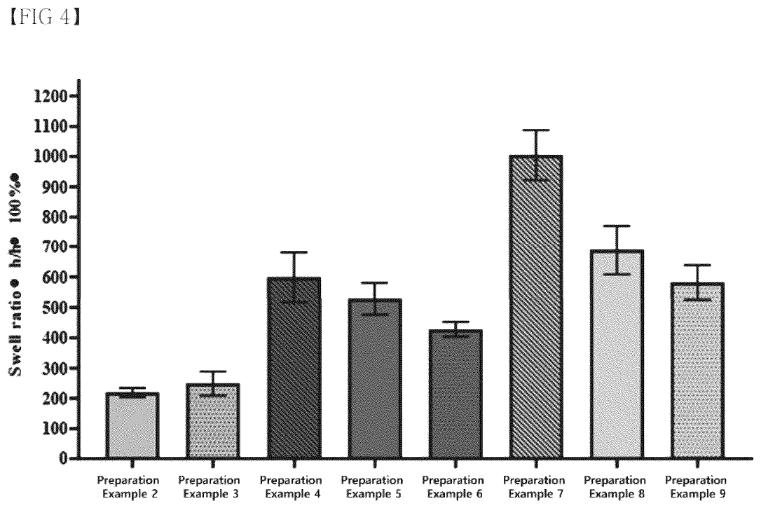

Preparation Example 2 and Preparation Example 3 were synthesized, respectively, by maintaining the content of chia seed mucilage at 0.5% and increasing the PVA content to 1% and 2%, respectively. However, the hydrogel films (Preparation Examples 2 and 3, respectively) failed to absorb water and to be swollen significantly in PBS (FIG. 3). This showed that when hydrogel films were prepared using 0.5% chia seed mucilage, the mechanical strength of the prepared hydrogel films was not sufficient and the gel performance was poor. As a result, the hydrogel films were not formed, and the water retention capacity of the hydrogel films was reduced. In the synthesis of hydrogel films using 1% or 2% chia seed mucilage, hydrogel films could be formed after drying, and the hydrogel films could absorb water and be swollen even when the PVA content was different. However, the properties of the hydrogel films were still different. The swelling heights of the preparation examples with 2% chia seed mucilage were in the range of 5.81 to 10.05 times that of the dry film. The swelling heights of the preparation examples with 2% chia seed mucilage were significantly higher than the swelling heights of the preparation examples with 1% chia seed mucilage, the swelling heights for 1% chia seed mucilage being in the range of 4.28 to 5.99 times that of the dry film (FIG. 4). However, this excessive expansion worsened the flexibility of the hydrogel films, made the hydrogel films impossible to maintain a stable shape in humid conditions, and made it easy for the hydrogel films to collapse and dissolve.

The moisture absorption rates of Preparation Examples 4 to 6 with 1% chia seed mucilage were uniform under PBS. Preparation Examples 4 to 6 enabled to stably absorb water and expand to maintain a uniform shape. However, Preparation Example 4 had insufficient adhesion and lost adhesion to the PE surface after being soaked in PBS for 2 hours. Preparation Example 6 has better adhesion than Preparation Example 5 after being immersed in PBS for 3 hours, but Preparation Example 6 experienced a flexibility decrease due to increased PVA content under dry conditions. Thus, Preparation Example 4 and 6 may not be suitable for joints with more activity. In addition, Preparation Example 5 was superior to Preparation Example 6 in moisture absorption rate and expandability.

Therefore, in the present disclosure, film-type wound dressing agents were prepared with 1% chia seed mucilage, 1% PVA, and 1.5% plasticizer each given hygroscopicity, flexibility, stability, and mechanical strength.

### 3. Properties of Film-Type Wound Dressing Agents

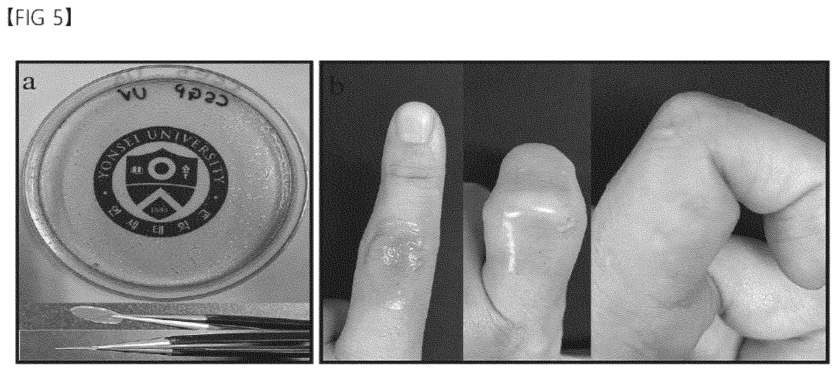

The size and thickness of the film-type wound dressing agents according to the present disclosure might be customized depending on the wound, and may also be prepared in a transparent form. At the same time, the film-type wound dressing agents might be directly attached to human skin and other objects by exhibiting unique self-adhesive properties. The film-type wound dressing agents might be directly attached to irregular surfaces such as finger joint creases, so the film-type wound dressing agents showed excellent deformation adaptability, and were able to withstand finger joint movement (FIG. 5). Additionally, there was no adhesive residue when the film-type wound dressing agents were attached to and removed from plastic, metal, glass and latex surfaces.

To intuitively confirm the adhesion and tensile strength of the film-type wound dressing agents, the prepared film-type wound dressing agents were attached to the wall of a 50 ml PE material centrifuge tube in a rectangular shape of 1 cm×5 cm. The weight of the centrifuge tube was adjusted by adding distilled water, the amount of which was in a range of 13 to 70 g, to the centrifuge tube.

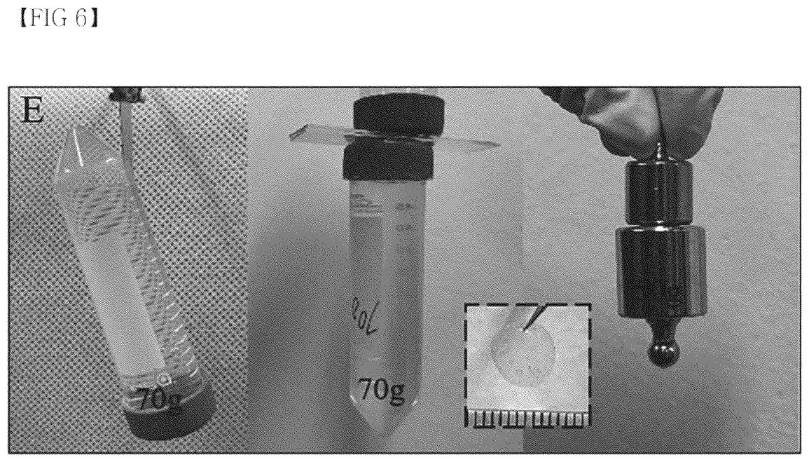

The tensile strength was calculated as follows, and as a result, the adhesive shear strength of the film-type wound dressing agents on the PE surface exceeded 1.4 kPa. Additionally, in experiments using circular film-type wound dressing agents with a diameter of 0.8 cm, it was confirmed that the peel strength of the circular film-type wound dressing agents at room temperature could exceed 14 kPa on a glass surface and 10 kPa on a metal surface (FIG. 6).

$$Tensile strength = Gravity ( g ) \times 0.0098 ( N / g ) / Area ( {m}^{2} )$$

As excess liquid was required to be absorbed from the wound area to keep the wound dry and prevent secondary infection, the liquid absorption ability of hydrogel films was crucial in wound dressings. Specimens of each film-type wound dressing agent were initially weighed and then immersed in PBS, the weights were measured over time, and the swelling ratio was calculated as follows. The swelling ratio of the film-type wound dressing agents increased over time in the PBS solution. It was observed that after 10 minutes of absorption of the PBS solution, the film-type wound dressing agents were able to absorb and retain twice the weight of the liquid. After 30 minutes, the maximum swelling ratio of 238% was reached.

$$Swelling ratio ( \% ) = ( Weight of film in swollen state - Weight of film in dry state ) \text{} / Weight of film in dry state \times 100$$

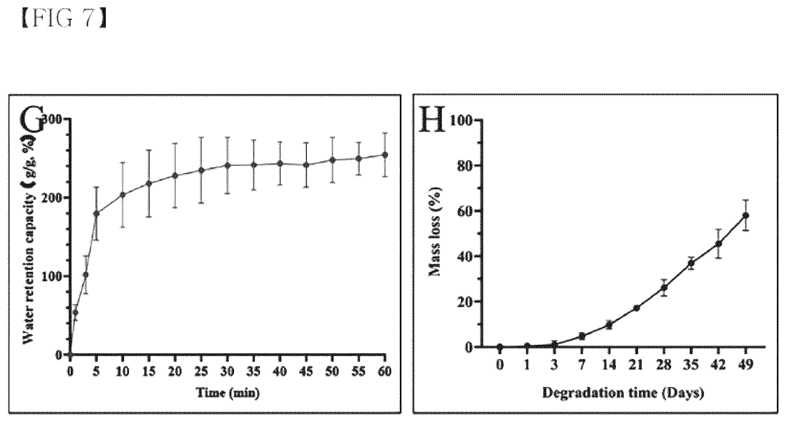

Meanwhile, as the polymer molecules expanded in the solvent, the chemical bonds between the molecules gradually weakened, and the film-type wound dressing agents decomposed after the film-type wound dressing agents were completely expanded in the PBS solution. 2 ml of PBS was added to each well of a 12-well cell culture plate to confirm the degradation rate. After weighing the initial weight of the film-type wound dressing agents (W1), the film-type wound dressing agents were fully saturated having been immersed in PBS for 1 hour, and then the film-type wound dressing agents were placed in a constant temperature and humidity incubator at a temperature of 37° C. for 45 days. After extracting solid samples from each well at different time intervals, the surface liquid was gently wiped off using the tip of a filter paper. Afterward, the solid residue was then dried and weighed (W2). This process was repeated three times, and the average weight was recorded. The decomposition rates of the samples were calculated as follows, and 50% of the weight of the film-type wound dressing agents was decomposed in the PBS solution at 45 days (FIG. 7).

$$Decomposition rate ( \% ) = ( W 2 - W 1 ) / W 1 \times 100$$

### 4. Antibacterial Effect of Pharmaceutical Composition for Wound Healing

The prepared film-type wound dressing agents were dried, weighed, cut into pieces, and placed in a high-speed shaker at a temperature of 37° C. PBS was added to the high-speed shaker. That was to make a completely dissolved 10 mg/mL pharmaceutical composition for wound healing.

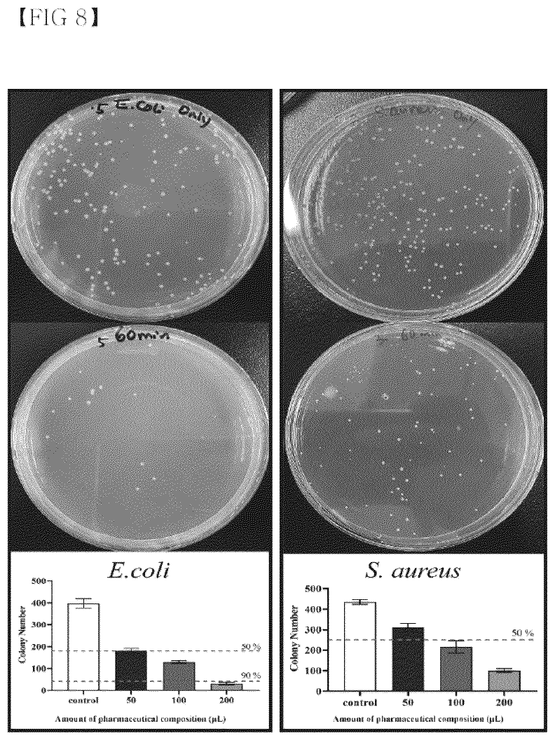

Escherichia coli (E. coli) and Staphylococcus aureus were each inoculated into a sterilized liquid medium and cultured at a temperature of 37° C. for 16 hours. After appropriately diluting the culture to 104 CFU/ml, 100 μl of the diluted bacterial solution was taken, added to a 1.5 ml centrifuge tube containing 100 μl of the 10 mg/ml composition, and mixed well. The mixture was left to stand for 15, 30, 45, and 60 minutes, respectively. Each mixture was then spread on the surface of a solid culture medium and transferred to 37° C. for 15 hours. The negative control test used PBS in which a film was not dissolved. The antibacterial activities of the composition against two pathogenic bacteria, Escherichia coli and Staphylococcus aureus, were evaluated using a total colony number. The pharmaceutical composition showed antibacterial effects against Escherichia coli and Staphylococcus aureus. In particular, in the case of E. coli, the growth of more than 90% of the bacteria was suppressed after 1 hour of mixing (FIG. 8).

### 5. In Vitro Healing Effect of Pharmaceutical Composition for Wound Healing

The biocompatibility of the pharmaceutical compositions for wound healing was tested on adult primary dermal fibroblast cells (HDFa). Fibroblast proliferation and migration in the outermost layer of the skin play an important role in tissue repair.

The prepared film-type wound dressing agents were dried, weighed, cut into pieces, and placed in a high-speed shaker at a temperature of 37° C. Fibroblast basal medium along with random FBS and growth factors was added to the high-speed shaker. That was to make a completely dissolved 25 mg/mL pharmaceutical composition for wound healing.

The HDFa cells were thawed at a temperature of 37° C. and cultured in the fibroblast basal medium containing 2% fetal bovine serum and fibroblast growth kit (HLL supplement: HSA 500 μg/ml, linoleic acid 0.6 mM, lecithin 0.6 μg/ml, L-glutamine: 7.5 mM, rh FGF basic: 5 ng/ml, rh EGF/TGF-1 supplement: 5 ng/ml and 30 μg/ml, rh insulin: 5 μg/ml, hydrocortisone: 1 μg/ml, ascorbic acid: 50 μg/ml). After adding different concentrations of the pharmaceutical composition for wound healing to the corresponding wells, the culture plate was incubated at a temperature of 37° C. and 5% CO₂ for 24 and 48 hours. After incubation, 10 μl MTT solution (5 mg/ml) was added to each well and incubated at a temperature of 37° C. for 4 hours. After removing the medium, 150 μl DMSO was added and mixed with a stirrer for 10 minutes. Afterward, absorbance was measured at a wavelength of 490 nm with a microplate reader.

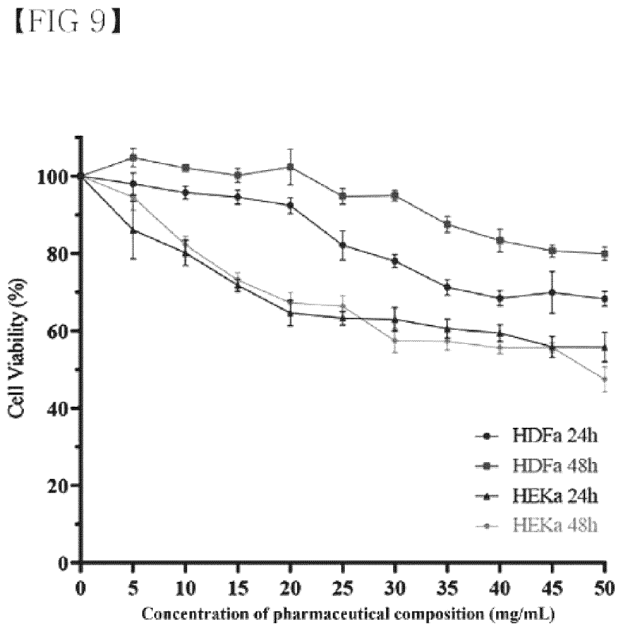

When the cells were cultured with less than 20 mg/ml of the pharmaceutical composition, there was no significant difference in the growth state of the cells compared to the control group. Additionally, the 48-hour culture group showed a greater increase in cell volume than the 24-hour culture group. Even in the 50 mg/ml group, the relative cell survival rate at 48 hours was still more than 80% (FIG. 9).

Meanwhile, a scratch assay was used to test the migration ability of the cells under the condition of the use of pharmaceutical composition for wound healing in vitro.

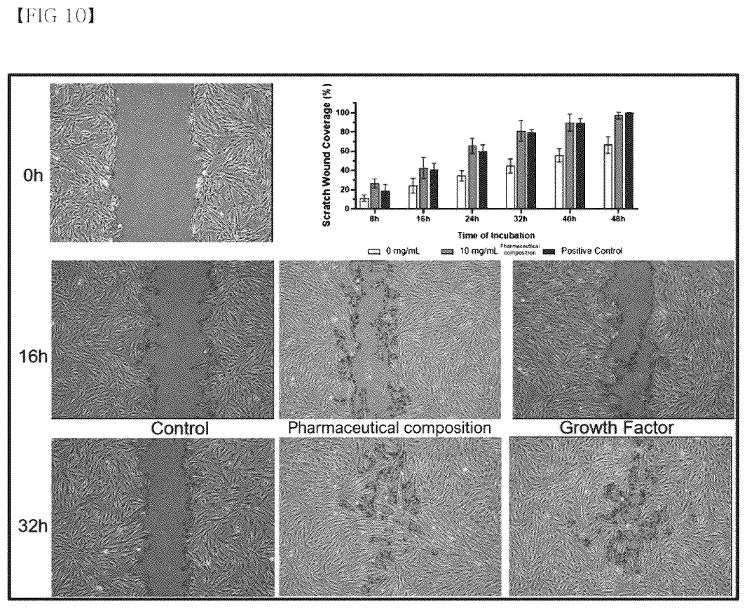

1×10⁵ cells/well were inoculated into a cell culture 6-well plate and cultured for 24 hours. Afterward, when the cells grew to 60% to 80%, the cell culture surface was scraped using a 1 ml pipette tip and a ruler. Excess cells floating on the surface were removed by gentle washing with PBS. The pharmaceutical composition and positive control solution (fibroblast basal medium containing 2% fetal bovine serum and fibroblast growth kit) were added at the corresponding concentrations. Cell migration at each time point in each group was observed using a microscope after 8, 16, 24, 32, 40, and 48 hours of culture. The scratch area and healed area at each time point in each group were calculated. The cell healing area subjected to the application of the pharmaceutical composition for wound healing was wider than that of the control group (no added serum, growth factors, or pharmaceutical composition). Within 24 hours after incubation, the pharmaceutical composition showed better healing ability than the positive control group (2% serum and growth factor supplementation). However, this advantage gradually decreased after 24 hours. After 48 hours, the scratches of each experimental and positive control group were healed (FIG. 10).

### 6. In Vivo Wound Healing Effect of Pharmaceutical Composition for Wound Healing

Female C57BL/6 mice, 8 to 10 weeks old, weighing approximately 24 to 30 g were purchased from Jackson Laboratory (Bar Harbor, ME, USA). The mice (n=5/group) were housed in a temperature (25° C.) and humidity (55±15%) controlled room in a pathogen-free facility at Yonsei University College of Medicine. The mice were exposed to a 12-hour light and 12-hour dark cycle. The upper back fur of the anesthetized mice was removed using an electric razor in accordance with hair removal protocol. All animal experiments were conducted in accordance with the ethical guidelines for animal research and were approved by the Institutional Animal Care and Use Committee of Yonsei University College of Medicine (IACUC No. 2020-0035). The mice were acclimatized for 1 to 2 weeks before starting experiments and monitored daily for general health. After anesthetizing the mice by having the mice inhale isoflurane (Hana Pharm, Seoul, Korea), two full-thickness fractures with a diameter of 0.8 cm were created on the back and buttocks along the spine of the mice. A wound without wound healing and that did not fall within the scope of wound healing application was used as a negative control. A film-type wound dressing agent was applied to the wound area of the experimental group. For comparison, DuoDerm® (ExtraThin CGF® dressing, ConvaTec, UK) was also used for testing. Images of entire wounds in the negative control group, DuoDerm® group, and film-type wound dressing agent group of the present disclosure were taken on days 0, 3, 7, 10, and 14.

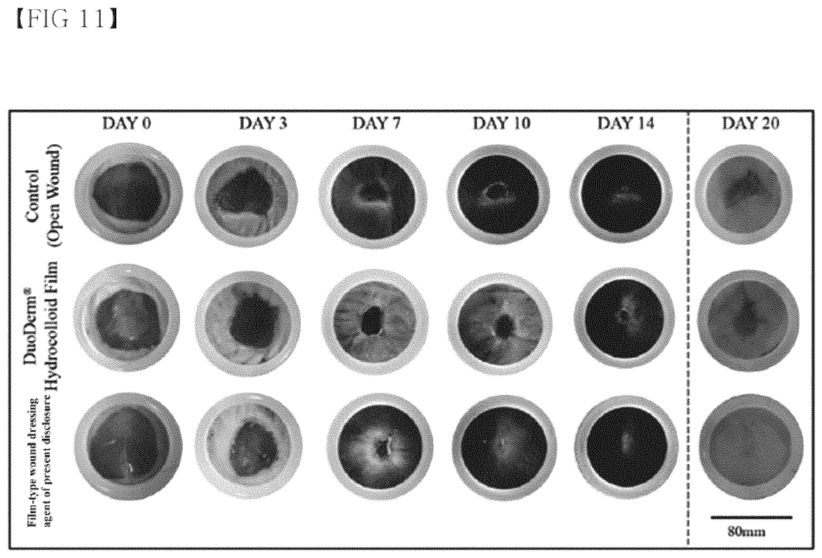

It was shown that the DuoDerm® film and the film-type wound dressing agent of the present disclosure allowed adequate moisture present on the wound surface and clean wound edge shrinkage. On day 7 of treatment, the untreated negative control group had some cracks in the wound area due to the dry healing state of continuous exposure. In both groups, the DuoDerm® film and the film-type wound dressing agent of the present disclosure continued to show effective healing and shrinkage. On day 10 of treatment, the film-type wound dressing agent of the present disclosure showed a better effect on wound healing than the other two groups. On day 14, the wound treated with the film-type wound dressing agent of the present disclosure was completely healed. Additionally, as a result of observing the condition of the hair follicles, it was found that the film-type wound dressing agent of the present disclosure was less harmful to wound healing and surrounding hair than the two groups. After all wounds had healed in all three groups, the scar condition of the wounds was evaluated, and the mice were shaved again. On day 20, the scar in the film-type wound dressing agent group were observed to be significantly smaller than those in the other two groups (FIG. 11).

Afterward, wound healing in each group was analyzed by applying ImageJ software (NIH, USA). The formula for calculating wound closure is as follows, where AO is the initial wound area measured on day 0 and Ad is the remaining wound area measured on days 3, 7, 10, and 14.

$$Wound closure ( \% ) = ( A 0 - Ad ) / A 0 \times 100$$

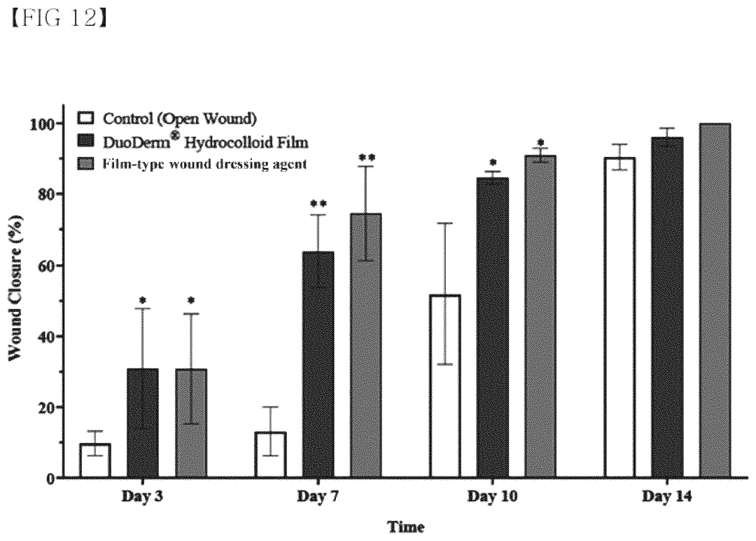

On day 3, the film-type wound dressing agent group of the present disclosure experienced a reduction in the wound area by 30.8%. This was higher than 30.9% in the DuoDerm® group and 9.7% in the untreated negative group. On day 7, the healing areas of the DuoDerm® film and the film-type wound dressing agent of the present disclosure were 63.9% and 74.6%, respectively. The treatment efficiency on day 10 was 85.6% in the film-type wound dressing agent group of the present disclosure, which was much higher than the negative control group (34.7%) (FIG. 12).

The wound healing potential of the film-type wound dressing agent was further evaluated using Hematoxylin-Eosin (HE) staining analysis on day 14. For examination, skin tissue was excised, preserved in a 4% neutral buffered formalin solution, embedded in paraffin, and cut into 4 mm sections. These sections were stained through hematoxylin-eosin and Masson's Trichrome staining. Tissue healing, epithelial cell morphology, and tissue structure were observed using an optical microscope.

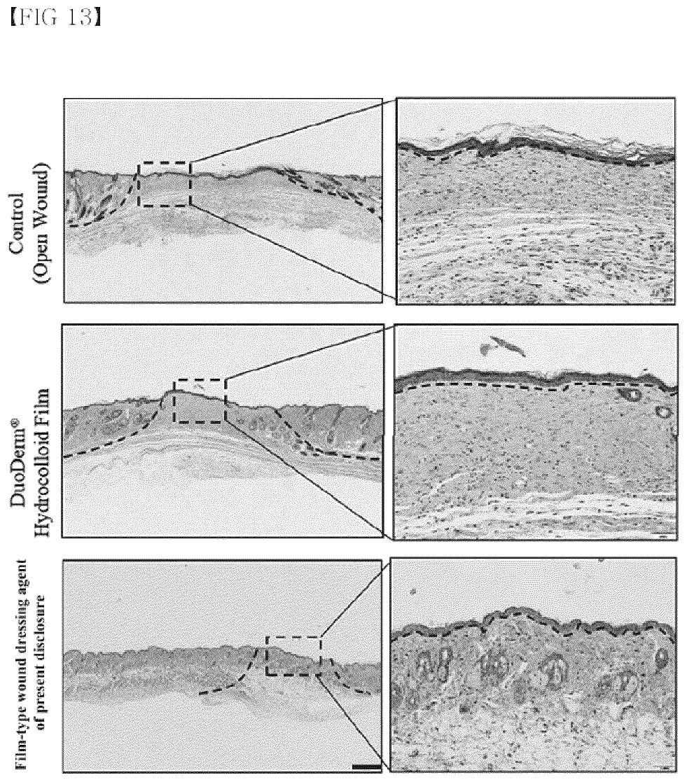

As a result of the analysis, complete re-epithelialization occurred in the film-type wound dressing agent group by 14 days. Epithelial regeneration and re-epithelialization occurred in all groups, but re-epithelialization occurred most readily in the film-type wound dressing agent group (FIG. 13).

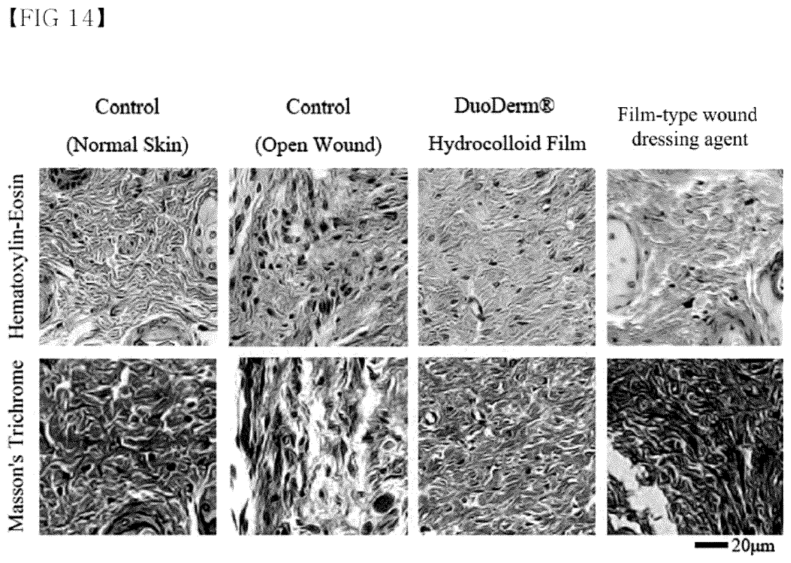

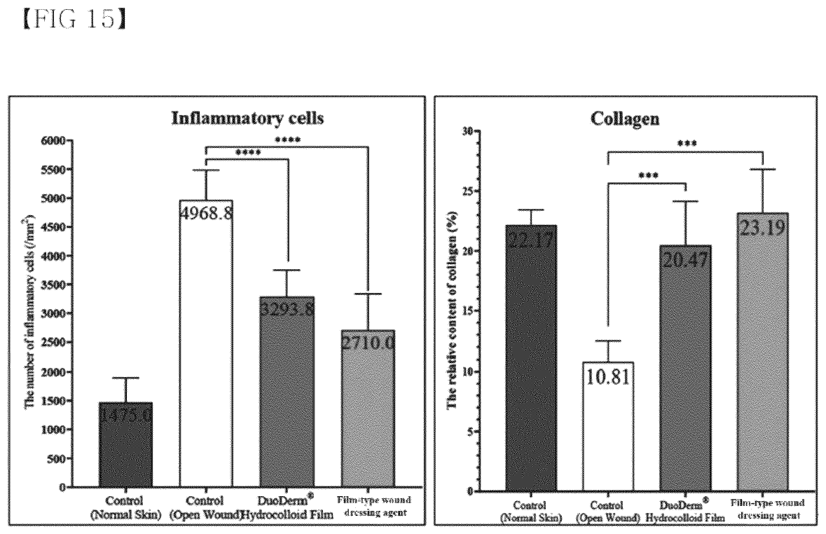

Additionally, further analysis was performed on the distribution of inflammatory cells in the wound areas and the regeneration of dermal tissue using high-resolution histology microscopy. It was confirmed that fewer inflammatory cells were significantly shown in the DuoDerm® and film-type wound dressing agent groups than in the negative control group. Thus, the DuoDerm® and film-type wound dressing agent groups were found to lower inflammation during the wound healing process, and the effect was greater in the film-type wound dressing agent group. Meanwhile, collagen stained blue through Masson's Trichrome staining indicated the level and distribution of collagen in healed skin. The presence of mature collagen tissue played a pivotal role in scar contraction, reepithelialization, and initial dermal organization. When the wound was treated with the film-type wound dressing agent of the present disclosure, more collagen staining was observed (FIGS. 14 and 15).

7. Analysis of mRNA and Biomarker Expression in Wound Tissue

To understand the wound healing effects of DuoDerm® and film-type wound dressing agent, four wound healing-related mRNAs (Fn1, Acta2, Col1a1, and TGFβ1), three angiogenesis-related mRNAs (Flk1, VEGFa, and Hif1a), and two inflammation-related mRNAs (Mmp9 and Nos2) were investigated. These mRNAs participated in various stages of wound healing from cell migration and proliferation to extracellular matrix (ECM) synthesis and tissue remodeling.

Total RNA was extracted from tissue powder and cell lysates using the AllPrep® DNA/RNA/Protein Mini kit (80004, Qiagen). Reverse transcription was performed using M-MLV reverse transcriptase (E-3122, Bioneer) and 1 μg of total RNA. The generated cDNA was diluted to a concentration of 0.5 μg/μl. Quantitative PCR analysis was performed on a Q real-time PCR cycler (Bio-Rad, Hercules, CA, USA) using AccuPower® 2× GreenStar™ qPCR Master Mix (Bioneer). Relative quantification of mRNA levels was performed using the comparative Ct method (ΔΔCt), and mRNA values were normalized to Actin. Additionally, immunohistochemical staining was performed using a DAB staining kit utilizing specific primary antibodies (Anti-αSMA, Anti-F4/80, and Anti-TGFβ markers). The resulting samples were evaluated using Cellsens software, and ImageJ software was used for the quantitative process.

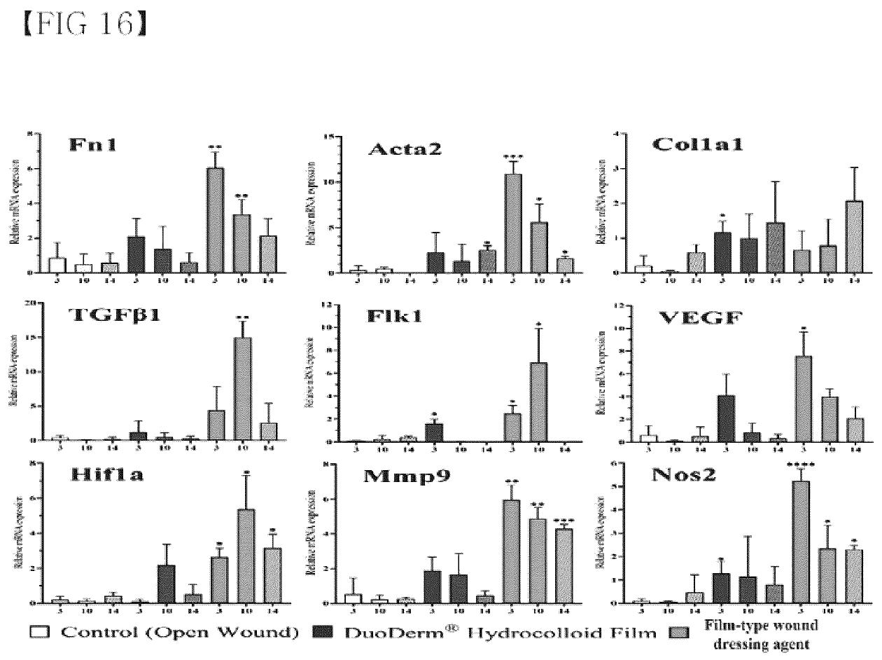

As a result, it was confirmed that the film-type wound dressing agent treatment increased the expression levels of mRNA related to wound healing, angiogenesis, and inflammation at all healing stages (days 3, 10, and 14), especially more than the negative control group. In particular, the film-type wound dressing agent group showed significantly higher mRNA levels of Fn1, VEGFa, Mmp9, and Nos2 compared to the DuoDerm® group and the control group, but these mRNA levels decreased over time. In the case of TGFβ1, Flk1, and Hif1a, the expression thereof peaked in the middle stage of wound healing and then decreased in the film-type wound dressing agent group. Additionally, Acta2 expression in the film-type wound dressing agent group surpassed that of the DuoDerm® group in the early and intermediate wound healing stages, while Col1a1 expression followed the opposite trend (FIG. 16).

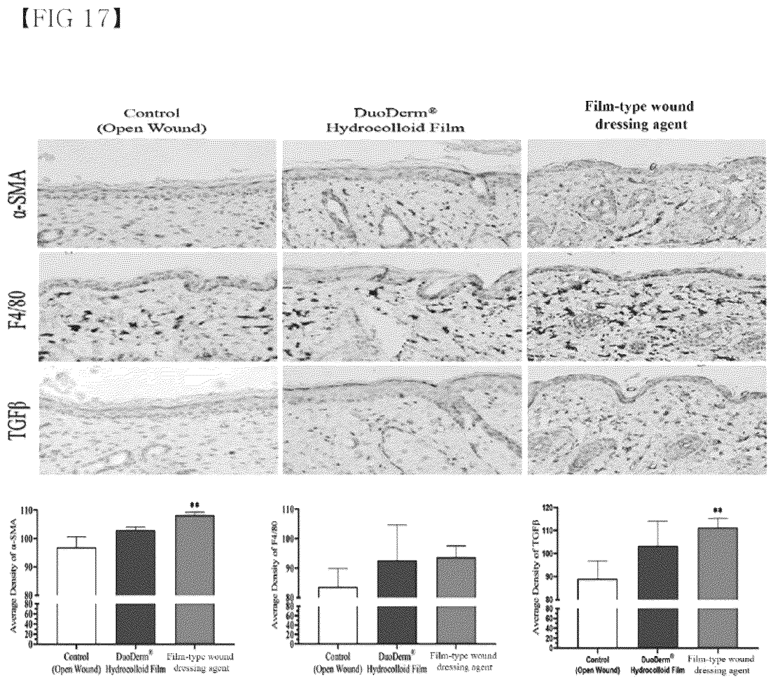

In addition, the results of immunohistochemical staining for α-smooth muscle actin (α-SMA), macrophage marker F4/80, and transforming growth factor beta (TGFβ) marker in the wound areas at day 14 further supported the healing potential of the film-type wound dressing agent. α-SMA was prominently expressed in granulation tissue during wound contraction and was a key identifier of mature myofibroblasts, enhancing tissue contraction and extracellular matrix (ECM) remodeling. F4/80 was a marker for macrophage differentiation and activation. TGFβ was crucial for wound healing because TGFβ promoted cell proliferation, differentiation, and ECM synthesis while controlling the activation state of immune cells. As a result of analyzing the densities of these three proteins, the protein density of the film-type wound dressing agent group exceeded those of both the negative control group and the DuoDerm group during the healing process (FIG. 17).

What is claimed is:

1. A method of antibacterial wound healing in a subject in need thereof, the method comprising administering an effective amount of a chia seed and a biodegradable substance.

2. The method of claim 1, wherein the chia seed is comprised in one or more forms selected from the group consisting of a hydrated chia seed shell, a swollen chia seed, and chia seed mucilage.

3. The method of claim 1, wherein the biodegradable substance comprises polyvinyl alcohol (PVA), sorbitol, and glycerol.

4. The method of claim 2, wherein the chia seed mucilage is contained in an amount of 0.5 to 2 w/v %.

5. The method of claim 3, wherein the PVA is contained in an amount of 0.5 to 2 w/v %.

6. The method of claim 3, wherein the sorbitol is contained in an amount of 1.5 w/v %.

7. The method of claim 3, wherein the glycerol is contained in an amount of 1.5 v/v.

8. The method of claim 1, wherein the chia seeds and the biodegradable substance are in a form of wound dressing agent.

9. The method of claim 8, wherein the wound dressing agent is formulated into one or more types selected from the group consisting of film, cream, gel, and spray.
